(19) United States
(12) Patent Application Publication
(10) Pub. No.: US 2021/0239696 A1
(43) Pub. Date: Aug. 5, 2021

(54) METHOD FOR MEASURING AND IMPROVING GUT HEALTH

(71) Applicant: CARBIOTIX AB, LUND (SE)

(72) Inventors: Peter FALCK, KAVLINGE (SE); Kristofer COOK, VIKEN (SE)

(21) Appl. No.: 17/054,089

(22) PCT Filed: May 9, 2019
(86) PCT No.: PCT/SE2019/050412

§ 371 Date: Nov. 9, 2020

(30) Foreign Application Priority Data
May 9, 2018 (SE) 1850550-3

(51) Int. Cl. G01N 33/569; G16H 20/10; G16H 20/60

(52) U.S. Cl. CPC G01N 33/56911; G01N 2469/10; G16H 20/60; G16H 20/10

(57) ABSTRACT

The present invention relates to a method for measuring gut health comprising the steps of: a) collecting or receiving at least one, preferably two faecal samples, preferably three or more faecal samples from a human or animal, and the sample comprises markers; b) using the sample of step a), to generate output databased on a composition and/or function and/or metabolic activity of gut microbiota; c) measuring output data in relation to level and/or stability of one or more marker sand/or relation between different markers to generate a result on gut health.

### FIELD OF THE INVENTION

The present invention relates to how the level, stability and relation between specific markers found in the microbiota can be used to measure and improve gut health. Further, the invention also describes how the results can be used to diagnose, prevent and treat disease. Further, the invention also describes how the results can be used to improve the quality of life. Further, the invention also describes how the results can be used to recommend a diet, lifestyle, supplement or medication.

### TECHNICAL BACKGROUND

There is a strong link between the gut microbiota (the billions of bacteria living in the gut) and different health conditions, weight gain, exercise, sleep, skin appearance and many other correlations being investigated. Measuring the composition or activity of gut bacteria has been suggested as a way to measure and improve gut health. These measurements can be the source of different diet recommendations or the development of drug candidates to treat disease.

Increasing the diversity of bacteria in the colon is seen as a beneficial contribution to gut health. However, it is lacking a good methodology to measure and improve gut health due to limitations in the current technology and understanding. The predictive power of microbiome data is very weak due to the to the large inter-individual differences and the day to day fluctuations in the microbiome. Therefore, up until now there is not a definition of a good or healthy microbiome.

Moreover, home testing which has been the foundation of many microbiome initiatives have the disadvantage of introducing errors in sampling and shipping. Therefore, it is unlikely that a single sample represents the dynamics of the individual's microbiome over time. Therefore, drawing conclusions based on one or a few samples does not take into account the nature of the microbiome as an adaptable ecosystem able to adapt to any change in diet, lifestyle or medication.

This invention is based on the discovery that individuals consuming a soluble fiber over a period of several months displayed an improvement in gut health based on different bacterial markers. Surprisingly the individuals displayed very different patterns of the bacterial markers. The individuals could be grouped into different groups based on their initial level of the markers and more importantly how the markers developed during the study. Surprisingly individuals having a low number of butyric acid producing bacteria initially responded by an increase in the marker while individuals having a higher initial value of the marker responded by a decrease in the marker.

This invention has the potential to revolutionize the methodology of testing gut health and also help with the diagnosis, prevention and treatment of chronic diseases such as cardiovascular disease, diabetes, obesity and cancer or improving quality of life for an individual. Once an optimal composition of the gut flora has been reach it can be used to monitor any change in the microbiota and link it to an early stage of a chronic disease.

### SUMMARY OF THE INVENTION

In one aspect, the present invention is a method for measuring gut health comprising the steps of:

a) collecting or receiving at least one, preferably two faecal samples, preferably three or more faecal samples from a human or animal, and the sample comprises markers;

b) using the sample of step a), to generate output data based on a composition and/or function and/or metabolic activity of gut microbiota;

c) measuring output data in relation to level and/or stability of one or more markers and/or relation between different markers to generate a result on gut health.

In one embodiment the method further comprises step d) using the result on gut health to diagnose, prevent, or treat disease.

In one embodiment the method of the present invention is to be performed on samples earlier obtained.

In another embodiment the method further comprise using the result on gut health to improve the quality of life, recommend a diet, lifestyle change, supplement or medication.

In one embodiment, the faecal samples are collected or received at a minimum once every 10 years, preferably every 5 years, preferably every 2 years, preferably every year, preferably every 6 months, preferably every 3^{rd} month, preferably every month, preferably every second week, preferably every week, preferably every day, preferably more than once a day. In one embodiment the fecal samples are collected or received at most every hour.

In one embodiment, the output data is generated by but not limited to Next Generation Sequencing (NGS), Whole-Genome Shotgun (WGS), Polymerase Chain Reaction (PCR), Quantitative or Real Time Polymerase Chain Reaction (RT-PCR or QPCR), DNA probe hybridization technology, Fluorescence in situ hybridization (FISH), High-Performance Liquid Chromatography (HPLC), Gas Chromatography (GC), Mass Spectrometry (MS), GC-MS, Nuclear Magnetic Resonance (NMR), Terminal-restriction fragment length polymorphism (T-RFLP), Denaturing Gradient Gel Electrophoresis (DGGE), Temperature Gradient Gel Electro-phoresis (TGGE). In one embodiment, the output data is composed of the absolute or relative quantities of at least one bacteria, gene and/or metabolite, preferably two, preferably three or more.

In one embodiment, the bacterium is a member of the microbiota. In one embodiment the bacterium is a member of the fiber degrading or metabolizing bacteria. In one embodiment the bacterium is belonging to Bacteroidetes, Firmicutes, Actinobacteria or Verrucomicrobia. Examples of such, but not limited to, may be Prevotella, Bacteroides, Akkermansia, Eubacterium, Bifidobacteria, Lactobacilli, Roseburia, Ruminococcus or Faecalibacterium. In one embodiment the bacterium is belonging to bacteria from Clostridial cluster IV or XIVa or Bifidobacteria. In one embodiment the bacteria are belonging to strains of Faecalibacterium prausnitzii and species of Bifidobacteria. Examples of such, but not limited to, may be Bifidobacterium adolescentis, Bifidobacterium longum, Bifidobacterium catenulatum, Bifidobacterium animalis, Bifidobacterium pseudolongum, Bifidobacterium gallicum, Bifidobacterium lactis, Bifidobacterium infantis, Bifidobacterium bifidum, Bifidobacterium angulatum or Bifidobacterium breve.

In one embodiment, the bacterium belongs to the group of mucus associated bacteria. In another embodiment the bacterium belongs to the core microbiome. In yet another embodiment, the bacterium belongs to short chain fatty acid producing bacteria. And in yet another embodiment the bacterium belongs to butyric acid producing bacteria.

In one embodiment, the marker is gene activity or metabolic activity displayed by any of the microbiota members. In one embodiment the marker is gene or metabolic activity related to fiber metabolism. In one embodiment it is related to short chain fatty acid biosynthesis. And in one embodiment it is related to propionic and/or butyric acid production.

In one embodiment, one marker is used in conjunction with another marker or markers. In one embodiment this is to monitor interactions between different bacteria. In another embodiment this is to monitor cross-feeding interactions. In yet another embodiment to monitor cross-feeding of organic acids such as short chain fatty acids and lactic acid between different bacteria. And in yet another embodiment to monitor the cross-feeding interaction between acetate and lactic acid producing bacteria and butyric producing bacteria. Examples of cross-feeding interactions may be between Bifidobacterium or Lactobacilli and Faecalibacterium prausnitzii.

In one embodiment the marker or markers is used in conjunction with markers of good gut health. Examples of such, but not limited to, may be the bacteria belonging to the group of Bacteroides such as Prevotella Porphyromonas, Alistipes, Parabacteroides and bacteria belonging to Christensenella and Clostridium scindens.

In one embodiment, the marker or markers is used in conjunction with markers of poor gut health. In one embodiment such markers may be pathogenic bacteria found in the microbiota. Examples of such, but not limited to, may be the bacteria belonging to the group of Escherichia, Salmonella, Shigella or C. difficile.

In one embodiment, the result is based on one, preferably two or more measurements of the level and/or stability of one or more markers and/or relation between different markers at each point of measurement.

In one embodiment, the result can be a sign of improving gut health, stable gut health or deteriorating gut health.

In one embodiment, the confidence level of the result increases with the number of samples.

In one embodiment, the result is grouped into different categories based on the starting values or patterns from one or more samples.

In one embodiment, the result is grouped into different categories based on results from an intervention with a fiber and/or probiotic and/or dietary change and/or lifestyle change to determine an individual's response and optimal gut health. In one embodiment, every result has an individual definition of optimal gut health. In one embodiment, the result is used to determine baseline and/or acute inflammation.

In one embodiment, a higher level and a more stable level of the marker or markers over time is indicative of a better gut health.

In one embodiment, a lower level and a more unstable level of the marker or markers over time is indicative of a poor gut health. In one embodiment, the interaction between different marker or markers is indicative of gut health.

In one embodiment, the results for different individuals is grouped based on different patterns of the marker or markers. In one embodiment, the patterns can be divided into three different phases, mobilization phase, growth phase and stabilization phase. In one embodiment, a change in the level and/or stability and/or relation between different markers is indicative of inflammation in the host. In one embodiment, the result is obtained by a trend analysis and/or fluctuation analysis, correlation analysis or statistical analysis of at least two samples, preferably three or more samples.

In one embodiment, the results are used to predict the outcome of an intervention with a fiber and/or probiotic and/or dietary change and/or lifestyle change and/or supplement and/or medication. In one embodiment, one marker is F. prausnitzii and/or Bifidobacterium. In one embodiment, the level of F. prausnitzii is used to indicate an optimal gut health in the intervals 50-0.1%, preferably 35-0.1%, preferably 20-0.5%.

In one embodiment, the level of Bifidobacterium is used to indicate an optimal gut health in the intervals 0.1-50%, preferably 1-50%, preferably 2-50%, preferably 5-50%, preferably 10-50%.

In one embodiment, the relation between F. prausnitzii and Bifidobacterium is used to indicate an optimal gut health and the ratio between F. prausnitzii and Bifidobacterium is between 100-0.01 preferably 50-0.02, preferably 25-0.04, preferably 10-0.1, preferably 5-0.2.

In one embodiment, a smaller standard deviation in the marker or markers in relation to the average value is used as a measure of gut health stability.

In one embodiment a high standard deviation is indicative of an unstable gut health. In one embodiment a low standard deviation is indicative of a more stable gut health. In one embodiment, the results on gut health is an indication of inflammation.

In one embodiment, a higher level and more stable gut health is more sensitive to the onset of inflammation.

In one embodiment, the inflammation is indicative of development or onset of metabolic or chronic disease.

In one embodiment , the results on gut health are used for diagnosis and/or prevention, and/or treatment of a disease, wherein the disease belongs to but is not limited to metabolic disease, gastrointestinal health, Crohn's disease, Ulcerative colitis, Multiple sclerosis, IBS, IBD, ADHD, Alzheimer's disease, muscle disease, non-alcoholic fatty liver disease, cardiovascular disease, allergy, asthma, diabetes, eczema and skin diseases, obesity, cancer, neurological health issues, endocrine system conditions, clostridium difficile associated conditions, locomotor system conditions, cutaneous condition, autoimmune system conditions, mental health associated conditions, skin related conditions, infectious disease and other health conditions associated with antibiotic usage, thyroid health issues, cerebro-craniofacial health, arthritis, dementia and kidney disease.

In one embodiment, the results on gut health are used for diagnosis and/or prevention, and/or treatment of a disease, wherein the disease belongs to Alzheimer's disease, cardiovascular disease, diabetes, cancer, and arthritis.

In one embodiment, the result is used to improve life, wherein the improvement is in more regular bowl movements, improved immune system, improved mineral absorption, improved cognitive function, improved glucose control, improved cholesterol control, improved triglycerides control, improved sleep, improved sex, improved weight control, improved weight loss, improved hair loss, reducing acne, improved skin appearance, improved muscle build, improved stamina, improved bone structure, improved teeth strength.

In one embodiment, the result used to base a recommendation, where in the recommendation is a healthier diet, preferably a more fiber-rich diet or a fiber supplement or a probiotic supplement or a medication.

In one embodiment, the recommended supplement fibers containing non- or partially digestible polysaccharides and/or oligosaccharides and/or disaccharides consisting of modified or unmodified starch and partial hydrolysates thereof, inulin or partially hydrolyzed inulin, natural oligofructoses, fructo-oligosaccharides (FOS), lactulose, lactosucrose, soybean-oligosaccharides (SOS), galactomannan and suitable partial hydrolysates thereof, manno-oligosaccharides (MOS), indigestible polydextrose, acemannan, various gums and pectin and partial hydrolysates thereof, indigestible dextrin and partial hydrolysates thereof, trans-galacto-oligosaccharides (GOS), xylo-oligosaccharides (XOS), xylan, arabinoxylan, arabinogalactan, arabino-xylooligosaccharides (AXOS), beta-glucan and partial hydrolysates thereof, chito-oligosaccharides (COS), glucomano-oligosaccharides (GMOS), arabinooligosaccharides (AOS), pectin-oligosaccharides (POS), laminar-oligosaccharides, human milk oligosaccharides (HMO), bovine milk oligosaccharides (BOS), cellulose derived oligosaccharides. Particularly, the recommended supplement fibers include arabino-xylooligosaccharides (AXOS), xylo-oligosaccharides (XOS), arabinoxylan, arabinogalactan. The recommended dose of the supplement fibers is 1-20 g per day. It is clear that the non-digestible or partially digestible fibers mentioned above is not limit to the examples given and can be of any origin or processed in a number of different ways.

### SHORT DESCRIPTION OF THE DRAWINGS

FIG. 1A. Relative percentage out of total bacteria of Faecalibacterium prausnitzii (FP). A clear convergence of a stable F. prausnitzii level is visible for all test subjects indicating a more diverse flora and improved gut health.

FIG. 1B. Relative percentage out of total bacteria of Bifidobacteria (BB). Almost half of the test subjects displayed a large increase in Bifidobacteria indicating a better gut health compared to the other test subjects.

FIG. 2A. Examples demonstrating the patterns and inter-individual difference in response to a soluble fiber and the measurement and improvement of gut health based on the relative amount of F. prausnitzii and Bifidobacteria and the ratio between the two reaching an optimal ratio between of 5-0.2. A. A relative decrease in F. prausnitzii and a delayed relative increase of Bifidobacteria.

FIG. 2B. Examples demonstrating the patterns and inter-individual difference in response to a soluble fiber and the measurement and improvement of gut health based on the relative amount of F. prausnitzii and Bifidobacteria and the ratio between the two reaching an optimal ratio between of 5-0.2. A. A stable level of F. prausnitzii and Bifidobacteria followed by a relative increase of Bifidobacteria.

FIG. 2C. Examples demonstrating the patterns and inter-individual difference in response to a soluble fiber and the measurement and improvement of gut health based on the relative amount of F. prausnitzii and Bifidobacteria and the ratio between the two reaching an optimal ratio between of 5-0.2. A. A relative decrease in F. prausnitzii and Bifidobacteria followed by a relative increase of Bifidobacteria.

FIG. 2D. Examples demonstrating the patterns and inter-individual difference in response to a soluble fiber and the measurement and improvement of gut health based on the relative amount of F. prausnitzii and Bifidobacteria and the ratio between the two reaching an optimal ratio between of 5-0.2. A. A relative decrease in F. prausnitzii followed by a relative increase and quick increase in Bifidobacteria followed by a relative decrease.

FIG. 3. One example of deteriorating gut health at which can be used to diagnose, prevent and treat different health conditions detected by an increase in the ratio between F. prausnitzii and Bifidobacteria. Caused by a relative decrease in Bifidobacteria in relation to F. prausnitzii from a state of stability.

### DETAILED DESCRIPTION OF THE INVENTION

Soluble fibers reach the colon where they are converted to short chain fatty acids to support the growth of a diverse flora of good bacteria, reducing the gut permeability and reducing inflammation. Therefore, an optimal gut health can be considered as a microbiota that has access to plenty of fiber through the diet. However, people are consuming too little fiber especially soluble fiber meaning there is a fiber deficiency and most people have not reach a state of optimal or even good gut health. There has been no definition of a normal, good or healthy microbiota due to the fact that most people have not optimized their microbiota with fiber through their diet.

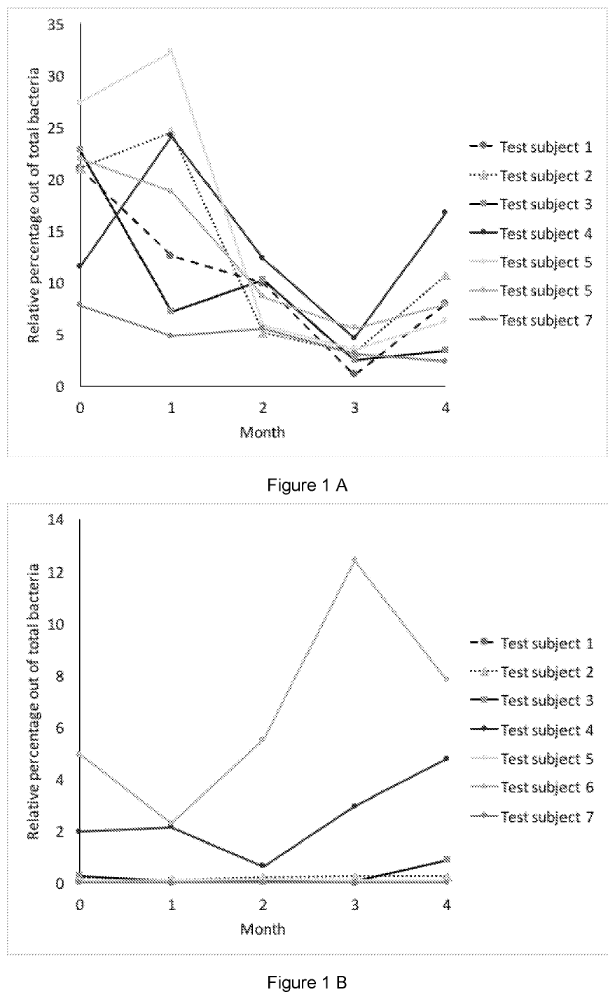

In order to address the problem of measuring and improving gut health for an individual, a measurement of different bacteria present in faecal samples was performed. A test group of 7 people took a soluble fiber supplement consisting of 2-5 g arabinogalactan every day for a total period up to 4 months. The test subjects sent in a fecal sample before starting the daily consumption of arabinogalactan. Bacterial DNA was extracted and QPCR was used to measure the level of total bacteria, F. prausnitzii, Bifidobacteria and Prevotella present in each sample. All results were stored together with each user's profile in a database. Based on the Ct values the relative percentage of each bacteria or bacteria group was determined (FIGS. 1A and 1B). The change of relative percentage of each bacteria or bacteria group as well as the ratio between different bacteria were used as a measure of gut health.

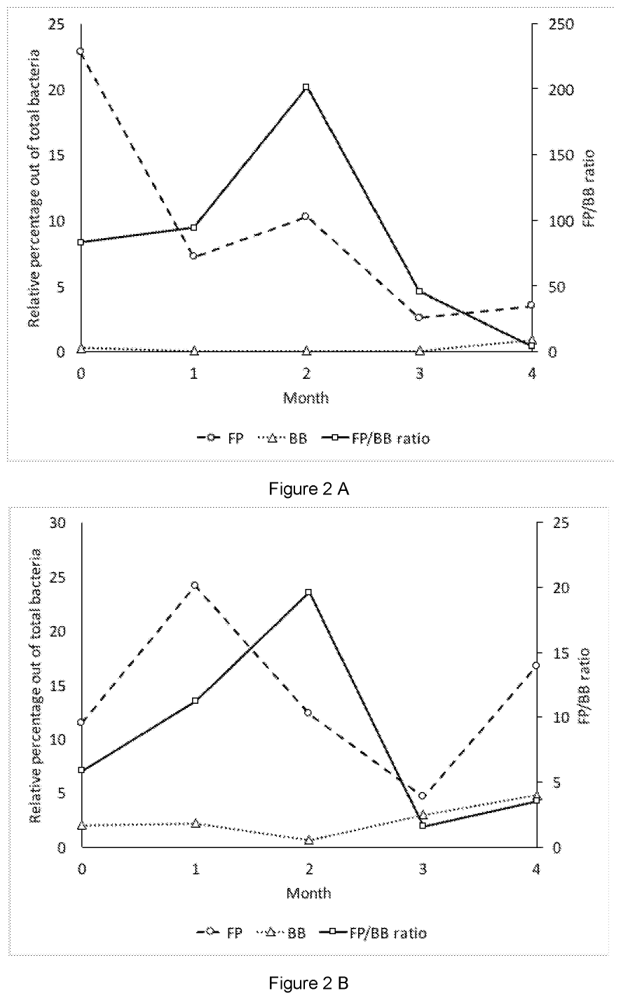

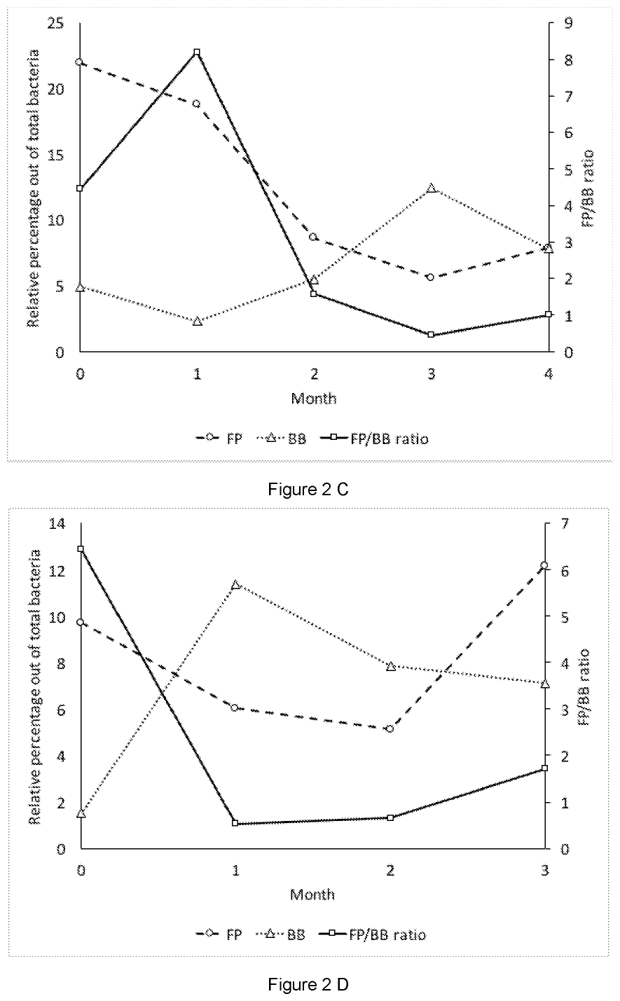

The pattern of butyric acid producing bacteria was only possible to discover due to the intervention with a soluble fiber known to increase bacterial diversity in the microbiota and following the microbiota development during several months. It took between 2 and 4 months to reach a stable microbiota depending on the individuals gut microbiome at the beginning of the study demonstrating that repeated measurements over a long period of time is important for measuring and improving gut health (FIGS. 2A, 2B, 2C and 2D).

Further, it was only possible to see change in the marker after increasing the fiber dose for some individuals showing the difficulties in discovering these patterns of gut health. Stabilizing the marker at an optimal level for gut health is therefore very individual based and should distinguish this invention from previous work in the area of microbiome diagnosis and drug development. The fact that it is possible with the current invention to measure an individual's response to an intervention or medication and to optimize their gut health makes it possible to classify an individual's microbiome as poor, sub optimized or optimized from a gut health perspective not possible before.

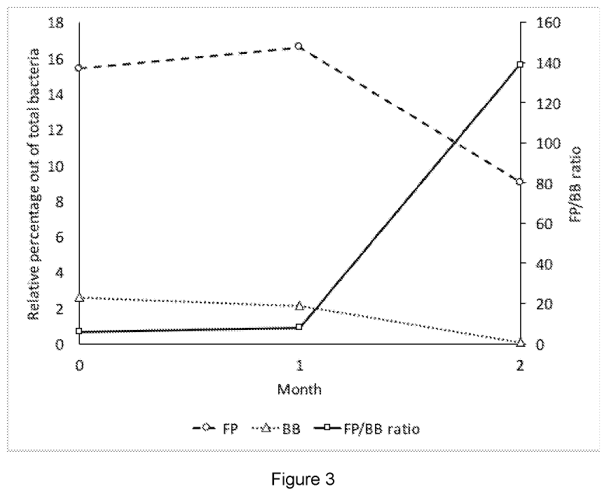

Furthermore, this invention demonstrates that by analysing the dynamics to discover patterns of bacteria in the microbiota after introducing a soluble fiber it was possible to draw conclusion about the impact on gut health of that particular individual. Moreover, the methodology can be used to monitor gut health once an individual has reach their optimal level of gut health due to the sensitivity of the microbiota to change in inflammation (FIG. 3).

It was surprising to discover that a single member of the core microbiome F. prausnitzii together with Bifidobacteria could be used as markers for both gut health, gut dysbiosis and a suboptimal gut flora. There is a clear correlation between the amount of fiber and the diversity of the microbiome and therefore the markers interplay discovered in this invention can be used as an indicator of bacterial diversity and gut health.

Surprisingly, even in otherwise healthy individuals there was an initial relative decrease of F. prausnitzii and/or Bifidobacteria by introducing fiber which shows a clear improvement in bacterial diversity making this method ideal for recommending diets, supplements, probiotics or medications for optimizing each individuals gut health. This is somewhat counterintuitively since a decrease F. prausnitzii and/or Bifidobacteria would usually be interpreted as something undesirable but with this current invention clearly demonstrates that a more normalized level of F. prausnitzii and/or Bifidobacteria to other gut bacteria can be more beneficial to gut health than simply a very high relative amount of one beneficial bacteria.

Reducing the amount of F. prausnitzii seems counterintuitive since it is regarded as a peace keeping bacteria or a new type of probiotic. It only makes sense in the light of keeping the absolute numbers constant while increasing other bacteria able to produce e.g. acetate to improve butyric acid production, diversity and bacteria load. Further, tracking a member of the core microbiome assuming it is rather constant such as F. prausnitzii, and the fluctuations is in the other microbes, makes it possible to correct or compensate for increasing total bacteria for e.g. measuring good probiotic bacteria impact in the total microbiome.

The results from the test group can be interpreted as three phases. The first phase with a relative decrease of the bacterial markers is indicative of a mobilization phase where the gut microbiota community of bacteria is adapting to a new environment of more available carbon sources in the form of fiber. This phase is then followed by a growth phase of good bacteria e.g. Bifidobacteria. Finally, there is a stabilization of the gut microbiota community at this new level of improved gut health called the stabilization phase.

How quickly an individual move between these three phases depends largely on the individual's unique microbiota as well as how much fiber is consumed through the diet. However, it is evident from the results that most people have the capacity to restore their microbiota and gut health through increasing their fiber intake.

Another surprising discovery was that by analysing multiple samples during a longer time frame it was possible to discover bacteria to bacteria interactions. For example, the cross-feeding with e.g. Bifidobacteria and butyrate producing bacteria belonging to clostridial clusters IV or XIVa. This cross-feeding produce butyric acid through breakdown of fibers to acetate by e.g. Bifidobacteria. Butyrate is well recognized as an important anti-inflammatory substance which helps to maintain a healthy gut barrier function. Measuring the cross-feeding and therefore the potential for short chain fatty acid production is another use of this method to monitor gut health together with monitoring core gut microbes.

For people, skilled in the art it should be obvious that other bacteria present in the microbiota could be used in a similar fashion as F. prausnitzii as a marker of gut health, flora diversity and fiber intake. However, F. prausnitzii belonging to the core microbiome (present in most individuals) and fairly stable during the life of a healthy individual would serve as one of the best markers of gut health. Further, F. prausnitzii is a single species making it ideal for detection with a fast and accurate detection methods such as QPCR. In people with disease it was possible to detect levels of bacteria with or without a fiber intervention that restored the gut microbiota composition to a healthy state. By measuring the levels of F. prausnitzii and Bifidobacteria , it was possible both to diagnose and treat conditions of IBD, diabetes, arthritis and cutaneous condition (Acne) (Table 1 A-D). It was surprising to find out that different levels of each biomarker was linked to a specific conditions and in the case of IBD and acne that the biomarker had to be tracked up to 3 months in order to see a pattern of unstability in these conditions, while in the case of diabetes and arthritis it was possible to with one sample see reduced levels of F. prausnitzii to diagnose and treated these two conditions. In the case of IBD and acne a stable level of Bifidobacteria and F. prausnitzii respectively was the indicator of a good treatment for each condition. Hence multiple samples are needed to diagnose and treat IBD and Acne since it is the stability of Bifidobacterium in the case of IBD and F. prausnitzii in the case of Acne which is not evident from only one sample. However, in the case if diabetes and arthritis a single sample seems to be enough to diagnose these conditions although multiple samples are needed to properly track and treat these conditions. The methods for measuring gut health herein described can be used for treatment of the subjects suffering from the diseases or syndromes herein described.

As mentioned above, the level of Faecalibacterium prausnitzii is used to indicate an optimal gut health in the intervals 50-0.1%. In one embodiment the level of Faecalibacterium prausnitzii is used to diagnose diabetes, where Faecalibacterium prausnitzii is less than 10%, more specifically less than 7%; or the level of Faecalibacterium prausnitzii is used in the treatment of diabetes, where Faecalibacterium prausnitzii level is increased to more than is more than 7%, specifically more than 10%, specifically more than 14%; or the level of Faecalibacterium prausnitzii is used to diagnose arthritis, where Faecalibacterium prausnitzii is less than 10%; or the level of Faecalibacterium prausnitzii used in the treatment of arthritis, where Faecalibacterium prausnitzii level is increased to more than is more than 10%, specifically more than 14%. Further, the level of Faecalibacterium prausnitzii is used to diagnose cutaneous condition, more specifically acne, where the level of Faecalibacterium prausnitzii is unstable and is reduced below 14%, more specifically in 3 month's time. The level of Faecalibacterium prausnitzii may also be used in the treatment of cutaneous condition, more specifically acne, where the level of Faecalibacterium prausnitzii is stabilized above 14%, more specifically in 3 month's time.

Also as mentioned above, the level of Bifidobacterium is used to indicate an optimal gut health in the intervals 0.1-50%. In one embodiment the level of Bifidobacterium is used to diagnose IBD, where the level of Bifidobacterium is unstable and is reduced below 4%, more specifically 3%, more specifically below 2%, more specifically in 3 month's time; or level of Bifidobacterium is used in the treatment of IBD, where the level of Bifidobacterium is stabilized above 2%, more specifically above 3%, more specifically above 4%;

The methods defined above includes that soluble fiber, more specifically soluble fiber mentioned as defined as the recommended fiber supplement is used to treat the aforementioned conditions.

### EXAMPLES

### Example 1: Measuring and Improving Gut Health in a Test Group After Intervention with a Soluble Fiber

Human fecal samples were collected from a test group of adult men and women every month up to 4 months including a base line sample taken before starting with the supplement. The fecal samples were bead beaten in a lysis buffer for 20 minutes. Bacterial DNA was isolated with magnetic beads and eluted in RNase free water. Total DNA was quantified using 260 nm using a nano-drop spectrophotometer. Quantitative PCR amplification and detection were carried out using primers for F. prausnitzii 5′-3′ (GGAGGAAGAAGGTCTTCGG & AATTCCGCCTACCTCTGCACT), Bifidobacteria 5′-3′ (CTCCTGGAAACGGGTGGT & GCTGCCTCCCGTAGGAGT), Prevotella 5′-3′ (CAGCAGCCGCGGTAATA & GGCATCCATCGTTTACCGT) and total bacteria 5′-3′ (ACTCCTACGGGAGGCAGCAGT & ATTACCGCGGCTGCTGGC),PCR amplification and detection was performed using an Quantstudio 3 (Applied Biosystems, Darmstadt, Germany) in optical-grade 96-well plates sealed with optical sealing tape. Each reaction mixture (22.5 μl) was composed of 10 μl of SYBR Green PCR Master Mix (Applied Biosystems, Darmstadt, Germany), 2 μl primer mix (10 pmol/μl each), 9 μl sterile distilled H2O, and 1.5 μl stool DNA (10 ng/μl). For the negative control, 2 μl of sterile distilled H2O was added to the reaction solution instead of the template DNA solution. A melting curve analysis was carried out following amplification to distinguish the targeted PCR product from the nontargeted PCR product. Each real-time PCRs were performed in triplicate, and average values were used for calculations. PCR conditions consisted of one cycle of 50° C. for 2 min, 95° C. for 2 min and then 40 cycles of 95° C. for 30s, 60° C. for 30s, and 72° C. for 60s. The fraction of each bacteria was calculated as 1/(2\hat{ }(delta Ct)), where delta Ct is the difference between the cycles for total bacteria and the target bacteria. The fraction of each bacteria was plotted in a graph to determine the pattern for each test subject. Example 2: F. prausnitzii and Bifidobacterium levels in people with IBD, diabetes, arthritis and cutaneous condition (acne) with or without an intervention with fiber. In order to find out what levels of F. prausnitzii and Bifidobacterium can be used to diagnose people with a disease, participants with IBD, diabetes, arthritis and cutaneous condition (acne) were tested for their levels of F. prausnitzii and Bifidobacterium with or without intervention with a soluble fiber (arabinogalactan). Fecal samples were collected, processed and analyzed as mentioned in example 1 up to 2 months with participants consuming non (control group) or up to 5 g per day of arabinogalactan (study group). The results average values were calculated for each bacterial marker for each group (with n participants) (Table 1A-D). It was possible both to diagnose and treat conditions by evaluating the level of each bacterial marker. For IBD Bifidobacterium was the best marker, however multiple tests were needed to detect the unstability in the level of Bifidobacterium in IBD cases (Table 1A). For diabetes F. prausnitzii was the best marker (Table 1B) where a low level of F. prausnitzii was evident in the control group vs. the fiber group. For Arthritis F. prausnitzii was the best marker (Table 1C) where a reduced level of F. prausnitzii was evident in the control group vs. the fiber group. For Acne F. prausnitzii was the best marker (Table 1D) where a reduced stability in the level of F. prausnitzii was evident from multiple samples in the control group vs. the fiber group. Table 1 A Relative percentages of F. prausnitzii (FP) and Bifidobacterium (BB) in a study group with people with IBD. Cont. is a control group with participants not consuming any fiber, while Fiber are participants consuming up to 5 g per day of soluble arabinogalactan fiber.

| | | IBD 0 | IBD 1 | IBD 2 | IBD |
|---|---|---|---|---|---|
| | | month | month | months | values |
| Cont. | FP % | 17.7 | 12.8 | 15.0 | 15.2 |
| (n = 22) | BB % | 5.1 | 3.7 | 1.8 | 3.5 |
| Fiber | FP % | 14.2 | 11.1 | 12.4 | 12.6 |
| (n = 57) | BB % | 5.8 | 4.2 | 4.4 | 4.8 |

Table 1 B Relative percentages of F. prausnitzii (FP) and Bifidobacterium (BB) in a study group with people with diabetes. Cont. is a control group with participants not consuming any fiber, while Fiber are participants consuming up to 5 g per day of soluble arabinogalactan fiber.

| | | Diabetes 0 | Diabetes 1 | Diabetes 2 | Average |
|---|---|---|---|---|---|
| | | month | month | months | values |
| Cont. | FP % | 5.1 | 7.3 | 8.1 | 6.8 |
| (n = 10) | BB % | 4.4 | 3.1 | 4.3 | 3.9 |
| Fiber | FP % | 10.4 | 16.1 | 16.4 | 14.3 |
| (n = 43) | BB % | 3.1 | 5.8 | 2.8 | 3.9 |

Table 1 C Relative percentages of F. prausnitzii (FP) and Bifidobacterium (BB) in a study group with people with arthritis. Cont. is a control group with participants not consuming any fiber, while Fiber are participants consuming up to 5 g per day of soluble arabinogalactan fiber.

| | | Arthritis 0 | Arthritis 1 | Arthritis 2 | Average |
|---|---|---|---|---|---|
| | | month | month | months | values |
| Cont. | FP % | 7.2 | 9.9 | 11.2 | 9.4 |
| (n = 16) | BB % | 5.9 | 4.6 | 4.7 | 5.1 |
| Fiber | FP % | 11.9 | 13.1 | 18.8 | 14.6 |
| (n = 56) | BB % | 4.9 | 2.6 | 2.7 | 3.4 |

Table 1 D Relative percentages of F. prausnitzii (FP) and Bifidobacterium (BB) in a study group with people with Acne. Cont. is a control group with participants not consuming any fiber, while Fiber are participants consuming up to 5 g per day of soluble arabinogalactan fiber.

| | | Acne 0 | Acne 1 | Acne 2 | Average |
|---|---|---|---|---|---|
| | | month | month | months | values |
| Cont. | FP % | 22.5 | 6.4 | 8.7 | 12.5 |
| (n = 20) | BB % | 7.5 | 2.9 | 5.1 | 5.2 |
| Fiber | FP % | 14.9 | 16.5 | 14.6 | 15.3 |
| (n = 50) | BB % | 7.9 | 4.0 | 4.0 | 5.3 |

1. Method for measuring gut health comprising the steps of:
a) collecting or receiving at least one, preferably two faecal samples, preferably three or more faecal samples from a human or animal, and the sample comprises markers;
b) using the sample of step a), to generate output data based on a composition and/or function and/or metabolic activity of gut microbiota;
c) measuring output data in relation to level and/or stability of one or more markers and/or relation between different markers to generate a result on gut health.

2. Method according to claim 1, further comprising the step:
d) using the result on gut health to diagnose, prevent or treat disease, improve the quality of life, recommend a diet, lifestyle change, supplement or medication.

3. Method according to claim 2, wherein the output data comprises quantities of at least one bacteria, gene and/or metabolite, preferably two, preferably three or more.

4. Method according to claim 3, wherein the at least one bacteria are a member of the microbiota, preferably member of the fiber degrading or metabolizing bacteria, preferably belonging to Bacteroidetes, Firmicutes, Actinobacteria or Verrucomicrobia.

5. Method according to claim 3, wherein the marker is gene activity or metabolic activity displayed by any of the microbiota members, preferably gene or metabolic activity related to fiber metabolism, preferably gene or metabolic activity related to short chain fatty acid biosynthesis, preferably gene or metabolic activity related to propionic and/or butyric acid production.

6. Method according to claim 1, wherein one marker is used in conjunction with at least one other marker, preferably to monitor interactions between different bacteria, preferably to monitor cross-feeding interactions, preferably cross-feeding of organic acids such as short chain fatty acids and lactic acid between different bacteria, preferably cross-feeding interaction between acetate and lactic acid producing bacteria, and butyric producing bacteria, preferably cross-feeding interaction between Bifidobacterium or Lactobacilli and Faecalibacterium prausnitzii.

7. Method according to claim 1, wherein the result is grouped into different categories based on results from an intervention with a fiber and/or probiotic and/or dietary change and/or lifestyle change to determine an individual's response and optimal gut health.

8. Method according to claim 1, wherein the result is used to determine a baseline and/or an acute inflammation.

9. Method according to claim 8, wherein a change in the level and/or stability and/or relation between different markers is indicative of inflammation in the host.

10. Method according to claim 4, wherein the level of Faecalibacterium prausnitzii is used to indicate an optimal gut health in the intervals 50-0.1%, preferably 35-0.1%, preferably 20-0.5%.

11. Method according to claim 4, wherein the level of Bifidobacterium is used to indicate an optimal gut health in the intervals 0.1-50%, preferably 1-50%, preferably 2-50%, preferably 5-50%, preferably 10-50%.

12. Method according to claim 6, wherein the relation between Faecalibacterium prausnitzii and Bifidobacterium is used to indicate an optimal gut health and the ratio between Faecalibacterium prausnitzii and Bifidobacterium is between 100-0.01 preferably 50-0.02, preferably 25-0.04, preferably 10-0.1, preferably 5-0.2.

13. Method according to claim 8, wherein the inflammation is indicative of development or onset of metabolic or chronic disease.

14. Method according to claims 1 and 13, wherein the results on gut health are used for diagnosis and/or prevention, and/or treatment of a disease, wherein the disease is selected from the group consisting of metabolic disease, gastrointestinal health, Crohn's disease, Ulcerative colitis, Multiple sclerosis, IBS, IBD, ADHD, Alzheimer's disease, muscle disease, non-alcoholic fatty liver disease, cardiovascular disease, allergy, asthma, diabetes, eczema and skin diseases, obesity, cancer, neurological health issues, endocrine system conditions, clostridium difficile associated conditions, locomotor system conditions, cutaneous condition, autoimmune system conditions, mental health associated conditions, skin related conditions, infectious disease and other health conditions associated with antibiotic usage, thyroid health issues, cerebro-craniofacial health, arthritis, dementia, and kidney disease.

15. Method according to claim 1, wherein the result is used to base a recommendation, where in the recommendation is a healthier diet, preferably a more fiber-rich diet or a fiber supplement or a probiotic supplement or a medication.

16. Method according to claim 1, wherein the recommended fiber supplement comprises non- or partially digestible polysaccharides and/or oligosaccharides and/or disaccharides consisting of modified or unmodified starch and partial hydrolysates thereof, inulin or partially hydrolyzed inulin, natural oligofructoses, fructo-oligosaccharides (FOS), lactulose, lactosucrose, soybean-oligosaccharides (SOS), galactomannan and suitable partial hydrolysates thereof, manno-oligosaccharides (MOS), indigestible polydextrose, acemannan, various gums and pectin and partial hydrolysates thereof, indigestible dextrin and partial hydrolysates thereof, trans-galacto-oligosaccharides (GOS), xylo-oligosaccharides (XOS), xylan, arabinoxylan, arabinogalactan, arabino-xylooligosaccharides (AXOS), beta-glucan and partial hydrolysates thereof, chito-oligosaccharides (COS), glucomano-oligosaccharides (GMOS), arabinooligosaccharides (AOS), pectin-oligosaccharides (POS), laminar-oligosaccharides, human milk oligosaccharides (HMO), bovine milk oligosaccharides (BOS), cellulose derived oligosaccharides.

17. Method according to claim 10, wherein the level of Faecalibacterium prausnitzii is used to diagnose diabetes, where Faecalibacterium prausnitzii is less than 10%, more specifically less than 7%.

18. Method according to claim 10, wherein the level of Faecalibacterium prausnitzii is used in the treatment of diabetes, where Faecalibacterium prausnitzii level is increased to more than is more than 7%, specifically more than 10%, specifically more than 14%.

19. Method according to claim 10, wherein the level of Faecalibacterium prausnitzii is used to diagnose arthritis, where Faecalibacterium prausnitzii is less than 10%.

20. Method according to claim 10, wherein the level of Faecalibacterium prausnitzii used in the treatment of arthritis, where Faecalibacterium prausnitzii level is increased to more than is more than 10%, specifically more than 14%.

21. Method according to claim 11, wherein the level of Bifidobacterium is used to diagnose IBD, where the level of Bifidobacterium is unstable and is reduced below 4%, more specifically 3%, more specifically below 2%, more specifically in 3 month's time.

22. Method according to claim 11, wherein the level of Bifidobacterium is used in the treatment of IBD, where the level of Bifidobacterium is stabilized above 2%, more specifically above 3%, more specifically above 4%.

23. Method according to claim 10, wherein the level of Faecalibacterium prausnitzii is used to diagnose cutaneous condition, more specifically acne, where the level of Faecalibacterium prausnitzii is unstable and is reduced below 14%, more specifically in 3 month's time.

24. Method according to claim 10, wherein the level of Faecalibacterium prausnitzii is used in the treatment of cutaneous condition, more specifically acne, where the level of Faecalibacterium prausnitzii is stabilized above 14%, more specifically in 3 month's time.

25. Method according to any claim 17-24 where soluble fiber, more specifically soluble fiber mentioned in claim 16 is used to treat the aforementioned conditions in claims 17-24.
